(19) United States
(12) Patent Application Publication
(10) Pub. No.: US 2019/0304591 A1
(43) Pub. Date: Oct. 3, 2019

(54) MEDICAL IMAGE MANAGEMENT DEVICE AND RECORDING MEDIUM

(71) Applicant: KONICA MINOLTA, INC., Tokyo (JP)

(72) Inventor: Makoto HORIUCHI, Suginami-ku (JP)

(21) Appl. No.: 16/361,557

(22) Filed: Mar. 22, 2019

(30) Foreign Application Priority Data
Mar. 27, 2018 (JP) 2018-059122

(51) Int. Cl. G16H 30/20; G16H 30/40; G16H 10/60

(52) U.S. Cl. CPC G16H 30/20; G16H 80/00; G16H 10/60; G16H 30/40

(57) ABSTRACT

A medical image management device includes a hardware processor that causes a mobile terminal to display a medical image thereon and transmits related information concerning the medical image via the mobile terminal at least in a part of a time period when the medical image is being displayed on the mobile terminal. The hardware processor varies a transmission target part of the related information over time.

### CROSS REFERENCE TO RELATED APPLICATIONS

The present application claims priority under 35 U.S.C. § 119 to Japanese Patent Application No. 2018-059122, filed on Mar. 27, 2018 is incorporated herein by reference in its entirety.

### BACKGROUND

### Technological Field

The present invention relates to a medical image management device and a recording medium.

### Description of the Related Art

Recently, information has been digitalized more and more in the medical field, and medical images, image interpretation reports, etc. are managed as digital data. Doctors interpret medical images which are being shown on a display device.

There has been a technique to show a medical image and an image interpretation report at the same time on respective screens for efficient reference to the medical image and the image interpretation report (see Japanese Patent Application Laid Open Publication No. 2009-223501), for example. The image interpretation report display screen is superimposed on the medical image display screen.

A medical imaging diagnosis support system is used to support input of a determination index showing a determination result of interpretation of an examination image (see Japanese Patent Application Laid Open Publication No. 2017-191519), in which an opinion input screen is shown on the examination image for input of opinions or determination index concerning the examination image.

For the regional medical cooperation or the improvement of work flow in a medical facility, mobile terminals such as tablet terminals and smartphones are used inside and outside the medical facility. It is reported that, advantageously, in a case where an emergency patient presents in a doctor's absence at the medical facility, a doctor may check medical images of the patient via the mobile terminal, while staying at home, etc. instead of getting to the medical facility.

However, the purpose, the past report(s), etc. of image interpretation included in the order received from the doctor who requested the image interpretation are often referenced during the interpretation of medical image. The image interpretation may be difficult on the mobile terminals such as tablet terminals or smartphones with a screen smaller in size than that of PCs (personal computers) in general, because the area for displaying the medical image may be limited as the related information such as the purpose and the past report(s) of image interpretation is displayed beside the medical image. The medical image display screen and other screens may be switched to one another, but such a method decreases the efficiency of the image interpretation.

In a technique in which an image interpretation report display screen or an opinion input screen is superimposed on a medical image display screen to keep the area for displaying a medical image as disclosed in Japanese Patent Application Laid Open Publication No. 2009-223501 and 2017-191519, the medical image is partially hidden and may not be observed as a whole.

### SUMMARY

The present invention is conceived in view of such problems in the conventional techniques, and has an object of giving related information concerning a medical image efficiently while maintaining the size of the medical image displayed on a mobile terminal.

To achieve at least one of the abovementioned objects, according to the present invention, a medical image management device reflecting one aspect of the present invention includes:

a hardware processor that causes a mobile terminal to display a medical image thereon and transmits related information concerning the medical image via the mobile terminal at least in a part of a time period when the medical image is being displayed on the mobile terminal,

wherein the hardware processor varies a transmission target part of the related information over time.

According to the present invention, a non-transitory computer-readable recording medium reflecting another aspect of the present invention in which a program is stored, wherein the program causes a computer cause a mobile terminal to display a medical image thereon, to transmit related information concerning the medical image via the mobile terminal at least in a part of a time period when the medical image is being displayed on the mobile terminal, and to vary a transmission target part of the related information over time.

### BRIEF DESCRIPTION OF THE DRAWINGS

The advantages and features provided by one or more embodiments of the invention will be more fully understood from the detailed description given hereinbelow and the appended drawings which are given by way of illustration only, and thus are not intended as a definition of the limits of the present invention.

FIG. 1 shows a system configuration of a medical imaging system in the first embodiment.

FIG. 2 is a block diagram showing a functional configuration of the medical image management device.

FIG. 3 is a block diagram showing a functional configuration of a client terminal.

FIG. 4 is a ladder chart showing an ordinary work flow in a medical imaging system.

FIG. 5 is a flow chart showing the first processing of providing a medical image executed by the medical image management device.

FIG. 6 shows an example of a medical image display screen displayed on a mobile terminal.

FIG. 7 is an explanatory image of transition of a transmission target part in display of a past image interpretation report on a mobile terminal.

FIG. 8A shows transition of related information to be displayed in a related information display area over time.

FIG. 8B shows the transition of the related information to be displayed in the related information display area over time.

FIG. 8C shows the transition of the related information to be displayed in the related information display area over time.

FIG. 9 is a flow chart showing the second processing of providing a medical image executed by a medical image management device in the second embodiment.

FIG. 10 is an explanatory image of transition of a transmission target part in voice output of a past image interpretation report on a mobile terminal.

### DETAILED DESCRIPTION OF EMBODIMENTS

Hereinafter, one or more embodiments of the present invention will be described with reference to the drawings. However, the scope of the invention is not limited to the disclosed embodiments.

### First Embodiment

First, the first embodiment of a medical image management device in accordance with the present invention is explained. The scope of the invention is not limited to the examples in the drawings.

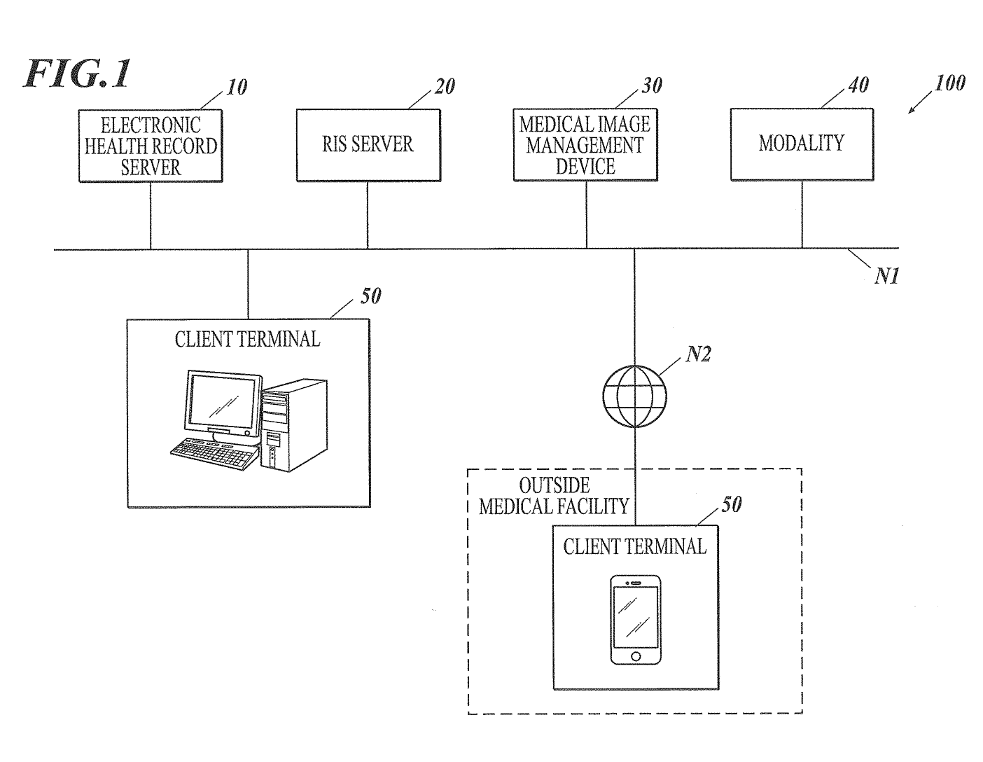

FIG. 1 shows a system configuration of a medical imaging system 100 in the first embodiment.

As shown in FIG. 1, the medical imaging system 100 is configured with a electronic health record server 10, an RIS (Radiology Information System) server 20, a medical image management device 30, a modality 40, a client terminal 50, etc. Each of the devices included in the medical image system 100 is connected to one another for data communication via an in-house network N1 such as a LAN (Local Area Network). Each of the devices is in conformity with the standards such as HL7 (Health Level Seven) and DICOM (Digital Image and Communications in Medicine), according to which the devices communicate with one another. The client terminal 50 outside the medical facility may be connected to the medical image management device 30, the electronic health record server 10, etc. for data communication via the internet N2. The number of the client terminals 50 is not particularly limited.

The electronic health record server 10 generates electronic health record information including a medical treatment(s) and a diagnosis result(s) concerning a patient, an examination order for requesting an examination of the patient, etc. according to the input operation from the client terminal 50.

The examination order includes a doctor who requested the examination (doctor in charge), an image interpretation doctor, patient information, examination information, etc.

The patient information is information on the patient. The patient information includes a patient ID, patient name, date of birth, age, sex, height, weight, blood pressure (systolic/diastolic), blood type, body temperature, history of present illness, past medical history, inpatient/outpatient classification, allergy record, infection record, etc.

The examination information is information on the examination. The examination information includes an examination ID, examination date, modality (DR, CR, US, CT, MRI, etc.), examined region (chest, abdomen, etc.), technician name, medical department, examination purpose, image interpretation purpose, use or non-use of contrast agent, reception number, etc.

The examination purpose is information from the person who requested the examination for informing the examination technician or the image interpretation doctor of the purpose of the examination.

The image interpretation purpose is information from the person who requested the examination for informing the image interpretation doctor of the purpose or the points of the image interpretation.

The RIS server 20 manages the information in the radiology department, such as the reservations of examination and treatment using the radiological devices and the examination results. The RIS server 20 manages an examination order(s) issued in the electronic health record server 10. The RIS server 20 then sends an examination request to the modality 40 used for the examination and sends an image interpretation request to the medical image management device 30 on the basis of the examination order.

The medical image management device 30 stores image data of the medical images generated in the modality 40 and manages the data for each patient. A PACS (Picture Archiving and Communication System) is used as the medical image management device 30, for example.

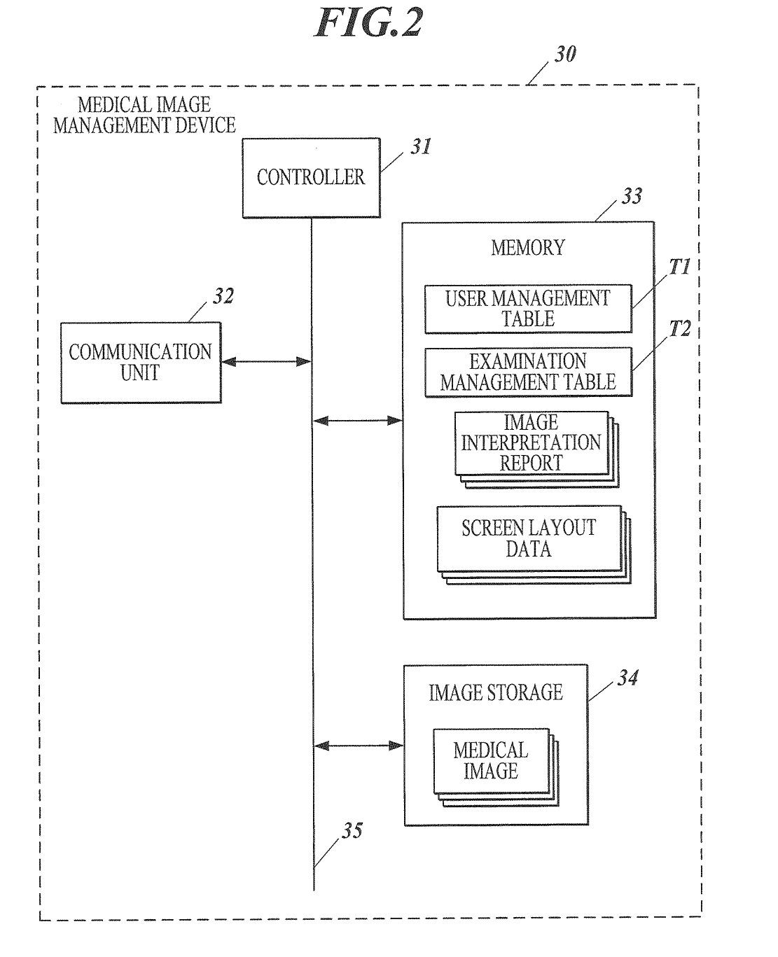

FIG. 2 shows a functional configuration of the medical image management device 30.

As shown in FIG. 2, the medical image management device 30 is configured with a controller 31, a communication unit 32, a memory 33, an image storage 34, etc., which are connected to one another by a bus 35.

The controller 31 is configured with a CPU (Central Processing Unit), a ROM (Read Only Memory), a RAM (Random Access Memory), etc. and integrally controls the processing operations of each unit of the medical image management device 30. Specifically, the CPU reads out various processing programs stored in the ROM and develops them in the RAM to perform the various kinds of processing in cooperation of the programs. The ROM stores therein a web server program to realize a function of a web server offering various web screens for the web browser by the communication between the web browser installed on the client terminal 50 and the HTTP protocol, an application program operating on the web server to provide information on medical images for the user of the client terminal 50 via the web browser, etc.

The communication unit 32 is configured with a network interface, etc., and sends and receives data to/from the external device connected via the in-house network N1 or the internet N2.

The memory 33 is configured with a hard disk, a non-volatile memory, etc. and stores various kinds of data therein. For example, a user management table T1, an examination management table T2, an image interpretation report(s), screen layout data, etc. are stored in the memory 33.

The user management table T1 is a table for managing users of the image management system of the medical image management device 30. The user management table T1 stores therein a user ID, password, name, where the user belong (medical facility, department), email address, etc. which are associated with each user (medical staff).

The examination management table T2 is a table for managing imaging examinations. The examination management table T2 stores therein a person who requested examination (doctor in charge), image interpretation doctor, patient information, examination information, identification information of medical image (image ID), identification information of image interpretation report (report ID), etc. which are associated with each imaging examination.

The image interpretation report is a report on an imaging diagnosis which is created according to the operation command from the client terminal 50 when the image interpretation doctor interprets a medical image on the client terminal 50. The image interpretation report is identified with the report ID.

The screen layout data is information on layouts of the medical image display screen (viewer screen), which specifies the arrangement of the area for displaying the medical image, the area for displaying the related information, etc. The screen layout data for mobile terminals (hereinafter referred to as layout for mobile terminal) and for PCs (hereinafter referred to as layout for PC) are prepared beforehand as the screen layout data. The mobile terminal is a portable information terminal device, such as a tablet and a smartphone, for example. There may be multiple layouts for PC prepared for respective configurations of monitor(s) (number of monitors, size of display screen, etc.) included in the client terminal 50.

The image storage 34 is a memory device for storing and preserving the image data of medical images of each patient therein. The medical images are identified with the image ID.

When a request to obtain the medical image or the image interpretation report is sent from the external device via the communication unit 32, the controller 31 sends screen data (data to be displayed) for displaying the requested medical image or image interpretation report to the external device according to the request.

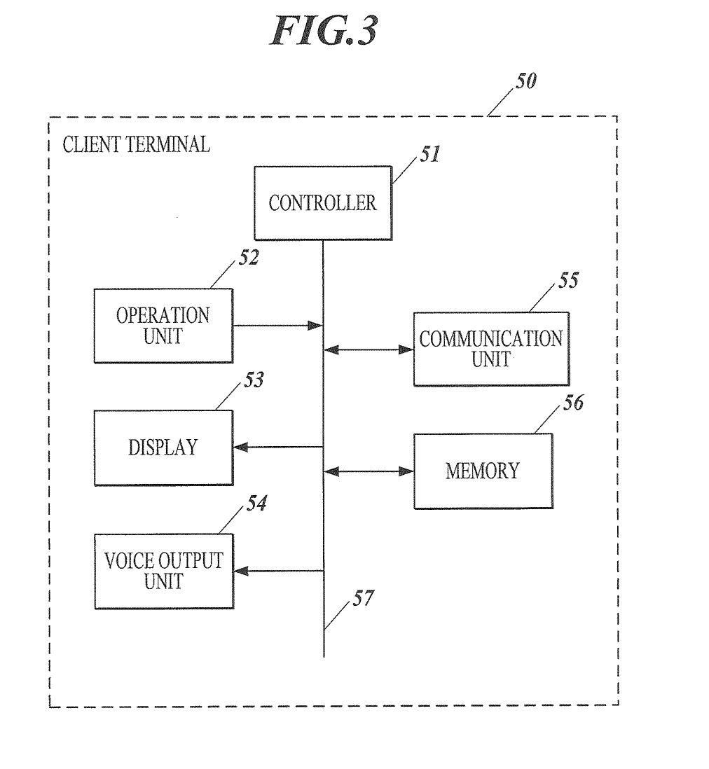

The controller 31 causes the client terminal 50 to show the medical image thereon. Specifically, the controller 31 sends the screen data for displaying the medical image on the display 53 (see FIG. 3) of the client terminal 50 via the communication unit 32 to the client terminal 50.

In a case where the client terminal 50 is a mobile terminal, the controller 31 transmits the related information concerning the medical image via the client terminal 50 at least in a part of the time period when the medical image is being displayed on the client terminal 50.

The controller 31 varies the transmission target part of the related information on the client terminal 50 over time. Specifically, the controller 31 does not set all the character strings included in the related information as the transmission target at the same time, but shifts the part to be transmitted in the character strings included in the related information over time.

The related information is attached information on the medical image, clinical information on the patient concerning the medical image, management information on the patient concerning the medical image, an instruction of the doctor concerning the medical image, a treatment plan of the patient concerning the medical image, or an image interpretation report created on a past examination corresponding to the examination concerning the medical image.

The “attached information on the medical image” is information attached to the medical image. The attached information is, for example, a patient name, patient ID, sex, examination ID, examination date and time, etc. included in the header of the image file of the medical image.

The “clinical information on the patient concerning the medical image” is information on consultation or treatment for the patient imaged as the target of the medical image. The clinical information includes, for example, a result of a biopsy such as uranalysis and blood analysis, etc.

The “management information on the patient concerning the medical image” is information on the management record in the medical facility concerning the patient imaged as the target of the medical image. The management information includes, for example, information on hospital admission, discharge, or transfer (medical facility, period, etc.), information on referral (medical facilities to or from which the patient is referred, details of referral, etc.), reservation of modality, etc.

The “instruction of the doctor concerning the medical image” includes, for example, an image interpretation purpose in respect of the interpretation of the medical image, an examination purpose in respect of the examination concerning the medical image, requirements and comments from the doctor who requested the interpretation of the medical image, etc.

The “treatment plan of the patient concerning the medical image” is information on treatment, medication, examination (examination order by diagnostic modality, planned biopsy, etc.), rehabilitation, etc. which are planned for the patient imaged as the target of the medical image.

The “past examination corresponding to the examination concerning the medical image” refers to a past examination identical to the examination concerning the medical image which has been performed on the patient imaged as the target of the medical image. Accordingly, the “image interpretation report created on a past examination corresponding to the examination concerning the medical image” is, in a case where an examination identical to the present examination has been performed on the patient imaged as the target of the medical image, an image interpretation report created from the interpretation of the medical image taken in the past examination.

In the explanation below, the past image interpretation report is mainly used as the related information. However, in a case where other information is used as the related information, the correspondence between the medical image to be displayed and its related information is registered beforehand inside or outside the medical image management device 30 and the medical image management device 30 may obtain and use the related information corresponding to the medical image to be displayed.

In the first embodiment, in a case where the client terminal 50 is a mobile terminal, the controller 31 causes the client terminal 50 to show the character strings of the related information in a predetermined area on the screen for displaying the medical image with scrolling. Specifically, the controller 31 generates the screen data for displaying the character strings of the related information in the predetermined area with scrolling while the medical image is being displayed, and provides the screen data for the client terminal 50 via the communication unit 32 to cause the client terminal 50 to display the screen data on the display 53. Here, the medical image may start to be displayed first and the related information with scrolling later, or vice versa. The medical image may cease to be displayed first and the related information with scrolling later, or vice versa. Accordingly, the period when the medical image is displayed on the display screen and the period when the related information is displayed with scrolling should overlap at least partially.

The modality 40 is an image generation device such as an X-ray imaging device (DR, CR), an ultrasound diagnostic device (US), CT, and MRI which generates medical images obtained by imaging of patients. The modality 40 writes the attached information (patient information, examination information, etc.) on the header of image file of the medical image to attach the attached information to the medical image in conformity with the standards of DICOM.

The client terminal 50 is a computer device used by a doctor such as a PC, tablet terminal, and smartphone. The doctor in charge creates an electronic health record, generates an examination order, and views the medical image or image interpretation report on the client terminal 50, for example. The image interpretation doctor interprets the medical image, and creates the image interpretation report on the client terminal 50, for example.

FIG. 3 shows a functional configuration of the client terminal 50.

As shown in FIG. 3, the client terminal 50 is configured with a controller 51, an operation unit 52, a display 53, a voice input unit 54, a communication unit 55, a memory 56, etc., which are connected to one another by a bus 57.

The controller 51 is configured with a CPU, a ROM, a RAM, etc., and integrally controls the processing operation of each unit of the client terminal 50. Specifically, the CPU reads out various processing programs stored in the ROM and develops them in the RAM to perform the various kinds of processing in cooperation with the programs. A web browser program to browse the Web is stored in the ROM, for example.

The operation unit 52 is configured with a keyboard including cursor keys, character and number input keys, function keys, etc., and a pointing device such as a mouse, and outputs, to the controller 51, the operation signals input by the key operation on the keyboard or the mouse operation. For the operation unit 52 configured with a touch panel layered on the display 53, the operation signals according to the positions of touch operation with user's fingers, etc. are output to the controller 51.

The display 53 is configured with a monitor such as an LCD (Liquid Crystal Display), and displays various screens thereon according to the command of the display signals input from the controller 51.

The voice output unit 54 includes a speaker, a D/A conversion circuit, etc. The voice output unit 54 converts the digital signals based on the voice data into the analog signals with the D/A conversion circuit, and outputs the voice on the basis of the analog signals from the speaker.

The communication unit 55 is configured with a network interface, etc., and sends and receives data to/from the external device(s) connected via the in-house network N1 or the internet N2.

The memory 56 is configured with a hard disk, a non-volatile memory, etc., and stores various kinds of data therein.

The controller 51 causes various screens to be shown on the display 53 on the basis of the screen data, etc. sent from the medical image management device 30.

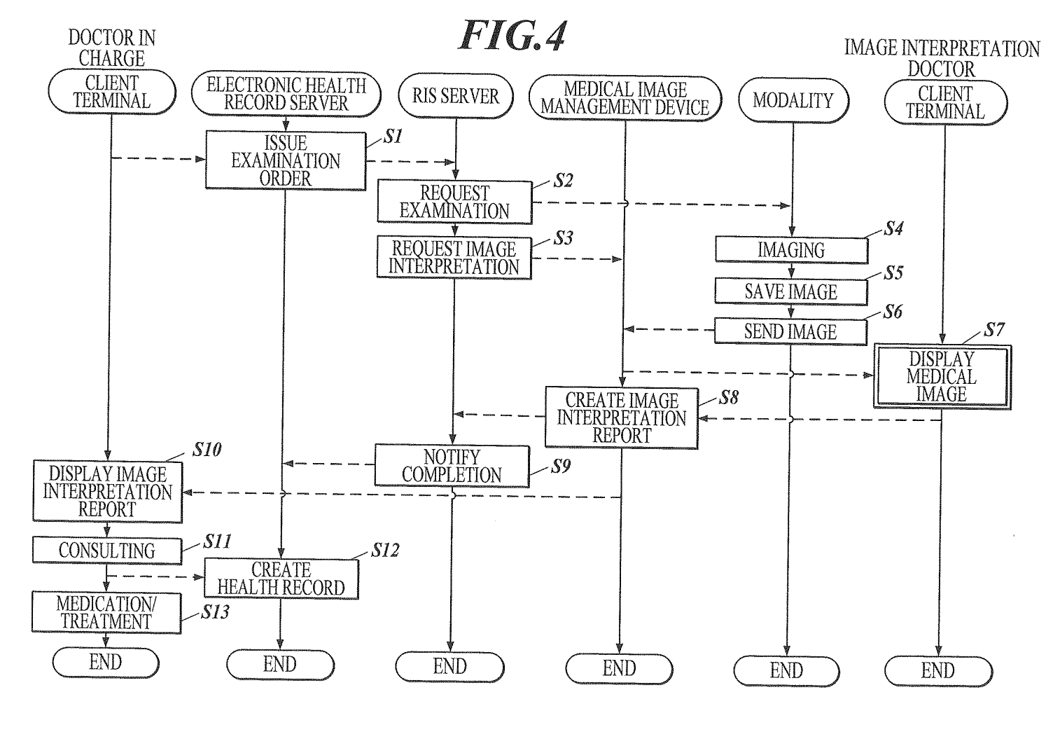

Next, the operation of the medical imaging system 100 is described. FIG. 4 is a ladder chart showing a general work flow in the medical imaging system 100.

First, the doctor in charge gets access to the electronic health record server 10 from the client terminal 50, and issues an examination order in which the patient and the examination details are specified (Step S1). The electronic health record server 10 sends the examination order to the RIS server 20.

The RIS server 20 sends an examination request to the modality 40 on the basis of the examination order received from the electronic health record server 10 (Step S2), and sends an image interpretation request to the medical image management device 30 (Step S3). The information to be included in the examination request or the image interpretation request may be selected from the information included in the examination order as needed. The examination order itself may be directly sent from the RIS server 20 to the modality 40 or the medical image management device 30.

The modality 40 performs imaging of the target region of the target patient according to the operation by the examination technician on the basis of the examination request in receipt of the examination request from the RIS server 20 (Step S4), and stores the generated medical image (Step S5). The modality 40 sends the medical image to the medical image management device 30 (Step S6).

In the medical image management device 30, in receipt of the medical image from the modality 40, the controller 31 specifies the image interpretation request corresponding to the medical image among the image interpretation requests received from the RIS server 20 on the basis of the attached information on the medical image, and the medical image and the specified image interpretation request (patient information, examination information, etc.) are associated to each other and saved. Specifically, the controller 31 stores the medical image in the image storage 34 and stores the person who requested the examination, the image interpretation doctor, the patient information, the examination information, and the image ID of the medical image, associating them to the examination management table T2 in the memory 33.

As the image interpretation doctor gets access to the medical image management device 30 from the client terminal 50 to select the medical image to be interpreted, the selected medical image is displayed on the display 53 of the client terminal 50 (Step S7).

The image interpretation doctor interprets the medical image displayed on the display 53 of the client terminal 50, inputs an image interpretation result with the operation unit 52, and creates an image interpretation report (Step S8). In the medical image management device 30, the controller 31 manages the created image interpretation report, associating it to the patient information and the examination information. Specifically, the controller 31 stores the image interpretation report in the memory 33 and stores the report ID of the interpretation report in the corresponding record in the examination management table T2. The medical image and the image interpretation report created on the medical image may be associated to each other as the person who requested the examination, the image interpretation doctor, the patient information, the examination information, the image ID of the medical image, and the report ID of the image interpretation report are associated in the examination management table T2.

Next, as the medical image management device 30 sends to the RIS server 20 a notification that the image interpretation is completed, the RIS server 20 sends to the electronic health record server 10 a notification that the image interpretation is completed (Step S9).

As the doctor in charge gets access from the client terminal 50 to the medical image management device 30 and gives a command to display the image interpretation report, the image interpretation report is displayed on the display 53 of the client terminal 50 (Step S10).

The doctor in charge examines the patient (Step S11), and creates the electronic health record with the access from the client terminal 50 to the electronic health record server 10 (Step S12). Specifically, the doctor in charge makes the treatment plan hereafter with reference to the image interpretation report and gives an explanation to the patient. The doctor in charge provides treatment and medication for the patient (Step S13).

For the processing of display and interpretation of the medical image at Step S7, a PC with a high resolution monitor inside the medical facility is usually used as the client terminal 50, but, in emergency, a mobile terminal outside the medical facility such as a tablet terminal or a smartphone is used as the client terminal 50. For example, as the doctor in charge (person who requests an examination) asks the image interpretation doctor for image interpretation by phone, the image interpretation doctor interprets the image on the mobile terminal, and, if necessary, asks the technician for an additional examination. If an additional examination is not particularly necessary, the image interpretation doctor informs the doctor in charge of the diagnosis content (image interpretation result) by phone or using a message tool of the image management system of the medical image management device 30. Contacted by the image interpretation doctor, the doctor in charge provides provisional treatment and medication for the patient, considering the diagnosis by the image interpretation doctor.

In a case where the image interpretation is simplified in emergency using the mobile terminal as described above, the processing from Step S7 is performed, for example, like a repeated image interpretation on PC while the image interpretation doctor is present at the medical facility.

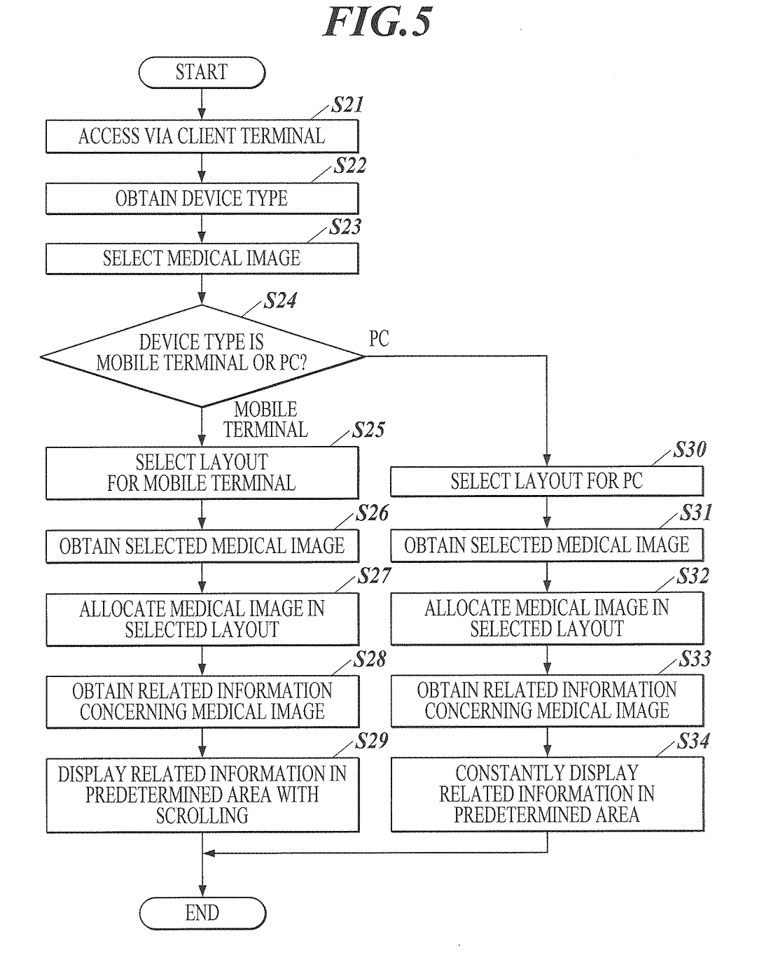

FIG. 5 is a flow chart showing the first processing of providing a medical image performed by the medical image management device 30. The processing is realized through the software processing by the CPU of the controller 31 in cooperation with the programs stored in the ROM.

The controller 31 first accepts access from the client terminal 50 via the communication unit 32 (Step S21). Specifically, as the user inputs the URL (Uniform Resource Locator) to access the medical image management device 30 on the Web browser via the operation unit 52 in the client terminal 50, the controller 51 connects to the medical image management device 30 via the communication unit 55 on the basis of the input URL.

In the medical image management device 30, the controller 31 provides screen data for displaying the login screen via the communication unit 32 for the client terminal 50.

The login screen is displayed on the display 53 in the client terminal 50. On the login screen, as the user inputs the user ID and password by the operation of the operation unit 52, the controller 51 sends the input user ID and password to the medical image management device 30 via the communication unit 55.

In the medical image management device 30, in receipt of the user ID and password via the communication unit 32, the controller 31 determines whether the received user ID and password correspond to any one of the combination of user ID and password registered in the user management table T1 of the memory 33, and performs processing of login.

Next, the controller 31 refers to the client device 50 for the device type via the communication unit 32, and obtains the type of the terminal device 50 (Step S22). Specifically, the controller 31 uses the API (Application Programming Interface) to refer to the client terminal 50 for the device type to obtain the device type. The device type may be mobile terminal, PC, etc. The PCs may be classified into multiple types according to the configuration of monitor (the number of monitors, the size of the display screen, etc.).

Next, the controller 31 receives the selection of the medical image to be displayed via the communication unit 32 from the client terminal 50 (Step S23).

The controller 31 determines here whether the device type obtained at Step S22 is mobile terminal or PC (Step S24).

If the device type is mobile terminal (Step S24; mobile terminal), the controller 31 selects a layout for the mobile terminal from the screen layout data stored in the memory 33 (Step S25).

Next, the controller 31 obtains the medical image selected at Step S23 from the image storage 34 (Step S26).

Next, the controller 31 allocates the medical image in the layout for mobile terminal selected at Step S25, and generates screen data for displaying the medical image display screen (Step S27).

Next, the controller 31 obtains the related information concerning the medical image (Step S28). For example, the controller 31 refers to the examination management table T2 in the memory 33 to specify the patient and examination associated to the image ID of the medical image to be displayed at present, and obtains the report ID of the image interpretation report concerning the past examination identical to the specified examination on the patient identical to the specified patient. The controller 31 then obtains the past image interpretation report stored in the memory 33 on the basis of the report ID. Alternatively, the controller 31 may obtain, from the memory 33, the image interpretation purpose or the examination purpose included in the image interpretation request concerning the medical image to be displayed at present. The controller 31 may, alternatively, obtain the image interpretation purpose or the examination purpose concerning the medical image to be displayed at present from the RIS server 20.

Next, the controller 31 generates the screen data for displaying the related information in the predetermined area of the medical image display screen with scrolling (Step S29). The controller 31 provides the generated screen data for the client terminal 50 (mobile terminal) via the communication unit 32.

In the client terminal 50 (mobile terminal), the medical image display screen is displayed on the display 53.

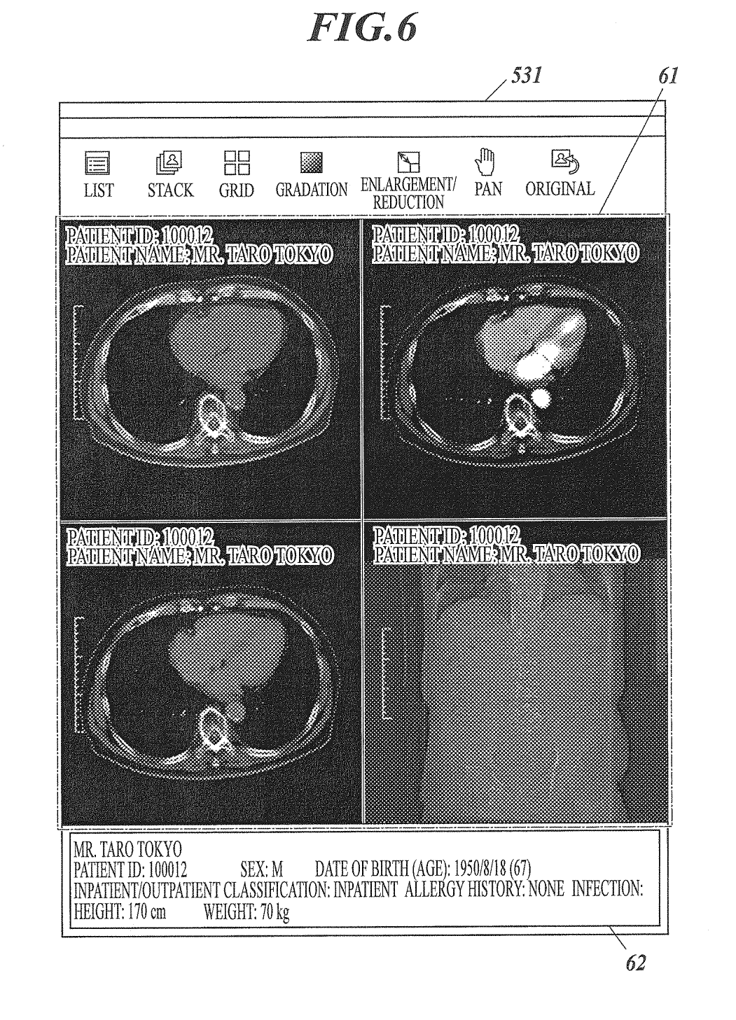

FIG. 6 shows an example of the medical image display screen 531 displayed on the display 53 of the client terminal 50 (mobile terminal). The medical image display screen 531 includes the medical image display area 61 and the related information display area (predetermined area) 62. The medical image display area 61 is an area for displaying the medical image, and the related information display area 62 is an area for displaying the related information. In the layout for mobile terminal, the related information display area 62 is set to be quite small in comparison to the medical image display area 61, so as not to obstruct the display of the medical image. The related information display area 62 is, for example, of such a size that one to five or so character strings may be displayed therein. The related information is displayed with scrolling from bottom to top in the related information display area 62.

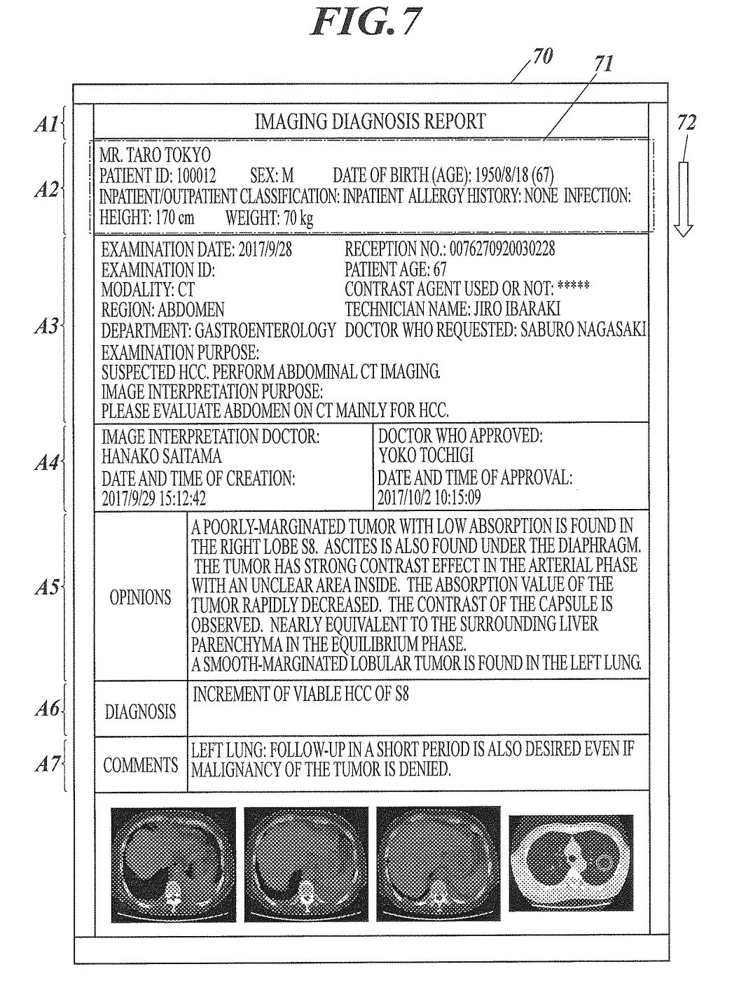

FIG. 7 shows an example of the past image interpretation report (imaging diagnosis report) 70. The information in the transmission target part 71 of the past image interpretation report 70 is displayed with scrolling in the related information display area 62 such that the transmission target part 71 boxed with a dashed line shown in FIG. 7 is shifted at a constant speed from top to the bottom (in the direction of the arrow 72 shown in FIG. 7). Specifically, the content in the areas A1 to A7 of the past image interpretation report 70 is successively displayed with scrolling.

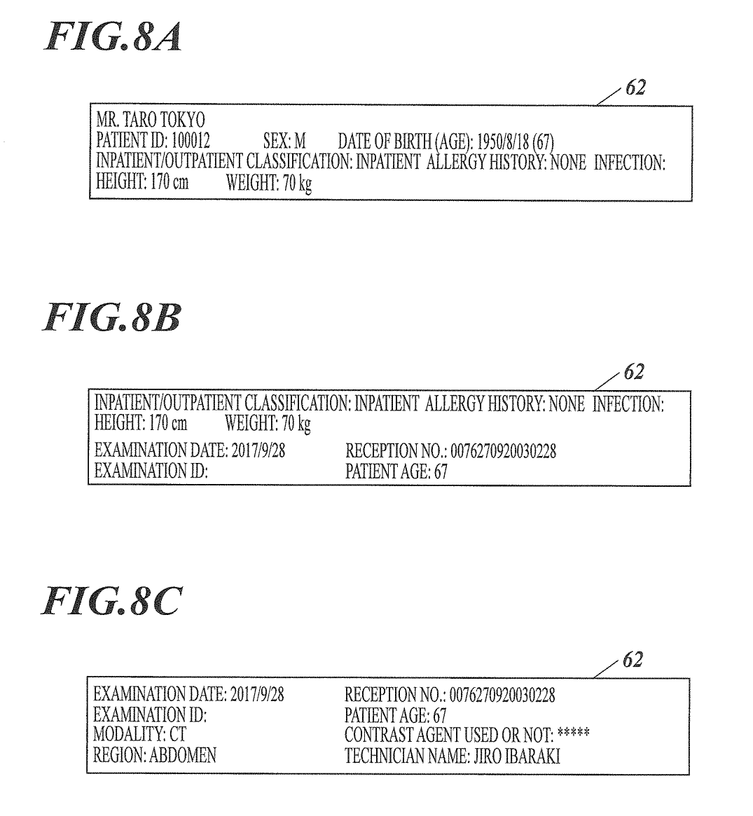

FIG. 8A to FIG. 8C show the successive transition of the related information to be displayed in the related information display area 62 over time. As the information in the past image interpretation report 70 is scrolled to the end, the scrolling stops there.

If the user decides to stop the scrolling of the related information, the scrolling of the related information stops as the user taps the related information display area 62 on the medical image display screen 531 shown in FIG. 6 by the operation of the operation unit 52.

If the user decides to check the information that has passed by, the scrolling may be adjusted. The part to be displayed turns back as the user swipes the related information display area 62 of the medical image display screen 531 shown in FIG. 6 in the direction opposite to the direction of scrolling by the operation of the operation unit 52, for example.

The position of start and end of scrolling or the item to be displayed with scrolling in the image interpretation report may be set for each facility or user, and the display arrangement of the image interpretation report may be switched depending on the medical facility where the users belong or depending on the user. The areas A1 to A7 shown in FIG. 7 are successively displayed with scrolling. Alternatively, the items may be displayed in a predetermined order with scrolling.

If the device type is PC at Step S24 (Step S24; PC), the controller 31 selects the layout for PC (Step S30).

Next, the controller 31 obtains the medical image selected at Step S23 from the image storage 34 (Step S31).

Next, the controller 31 allocates the medical image in the layout for PC selected at Step S30, and generates screen data for displaying the medical image display screen (Step S32).

Next, the controller 31 obtains the related information concerning the medical image (Step S33).

Next, the controller 31 generates the screen data for displaying constantly the related information in the predetermined area of the medical image display screen (Step S34). The controller 31 provides the generated screen data for the client terminal 50 (PC) via the communication unit 32.

In the client terminal 50 (PC), the medical image display screen is displayed on the display 53. The medical image display screen includes the medical image display area and the related information display area (predetermined area). The related information display area of the layout for PC is of such a size that all the related information is displayed at the same time. The related information may be displayed partially also in the layout for PC, if required by the size of the related information display area and the amount of the related information.

After Step S29 or S34, the first processing of providing a medical image is finished.

As described above, according to the first embodiment, in a case where the client terminal 50 is a mobile terminal, the transmission target part of the related information concerning the medical image is varied over time. Thus, the space for displaying all the related information at the same time is not necessary. Accordingly, the medical image may be displayed in a sufficient size on the client terminal 50 while the related information concerning the medical image may be efficiently transmitted at the same time.

Specifically, in a case where the client terminal 50 is a mobile terminal, the character strings of the related information are displayed with scrolling in the predetermined area on the screen for displaying the medical image in the client terminal 50, thus the related information may be transmitted without the display of medical image being obstructed.

On the other hand, in a case where the client terminal 50 is a PC, both the medical image and the related information are displayed at the same time so that the user may get the information in full view.

In the first embodiment, on the display 53 of the client terminal 50 (mobile terminal), the related information is displayed with scrolling in the vertical direction (from bottom to top) of the screen. However, the related information may be displayed with scrolling in the horizontal direction (from right to left) of the screen.

On the display 53 of the client terminal 50 (mobile terminal), the related information is displayed with scrolling. Alternatively, the related information may be separated into multiple fields and displayed by each of the separated fields which are switched to one another by a predetermined period of time.

### Second Embodiment

Next, the second embodiment in accordance with the present invention is described. The medical imaging system in the second embodiment is configured in the same way as the medical imaging system 100 described in the first embodiment. Thus, FIG. 1 to FIG. 3 are cross-referred to and the configurations are not shown by illustration or explanation. Hereinafter, the configurations and processing characteristic of the second embodiment are described.

In the first embodiment, the related information concerning the medical image is displayed with scrolling in a case where the client terminal 50 is a mobile terminal. In the second embodiment, however, the related information concerning the medical image is output in a voice in a case where the client terminal 50 is a mobile terminal.

The controller 31 of the medical image management device 30 causes the client terminal 50 to display the medical image thereon.

In a case where the client terminal 50 is a mobile terminal, the controller 31 transmits the related information concerning the medical image via the client terminal 50 at least in a part of the time period when the medical image is being displayed on the client terminal 50.

The controller 31 varies the transmission target part of the related information over time.

In a case where the client terminal 50 is a mobile terminal, the controller 31 generates voice data by converting the character strings of the related information into voice and, on the basis of the voice data, causes the voice output unit 54 of the client terminal 50 to output the voice. The conversion into voice is processing in which the character strings (text data) are converted into voice data, and a known software of text-to-speech may be used for the processing. Here, the display of the medical image may start first and the voice output of the related information later, or vice versa. The voice output of the related information may be terminated first and the display of the medical image later, or vice versa. Accordingly, the period when the medical image is displayed on the display screen and the period when the related information is output in a voice should overlap at least partially.

In the second embodiment, among the screen layout data stored in the memory 33, the layout for mobile terminal may not include an area for displaying the related information.

The controller 51 of the client terminal 50 causes the display 53 to display various screens thereon on the basis of the screen data sent from the medical image management device 30, etc.

The controller 51 causes the voice output unit 54 to output the related information in a voice on the basis of the voice data sent from the medical image management device 30.

Next, the operation in the medical imaging system in accordance with the second embodiment is described.

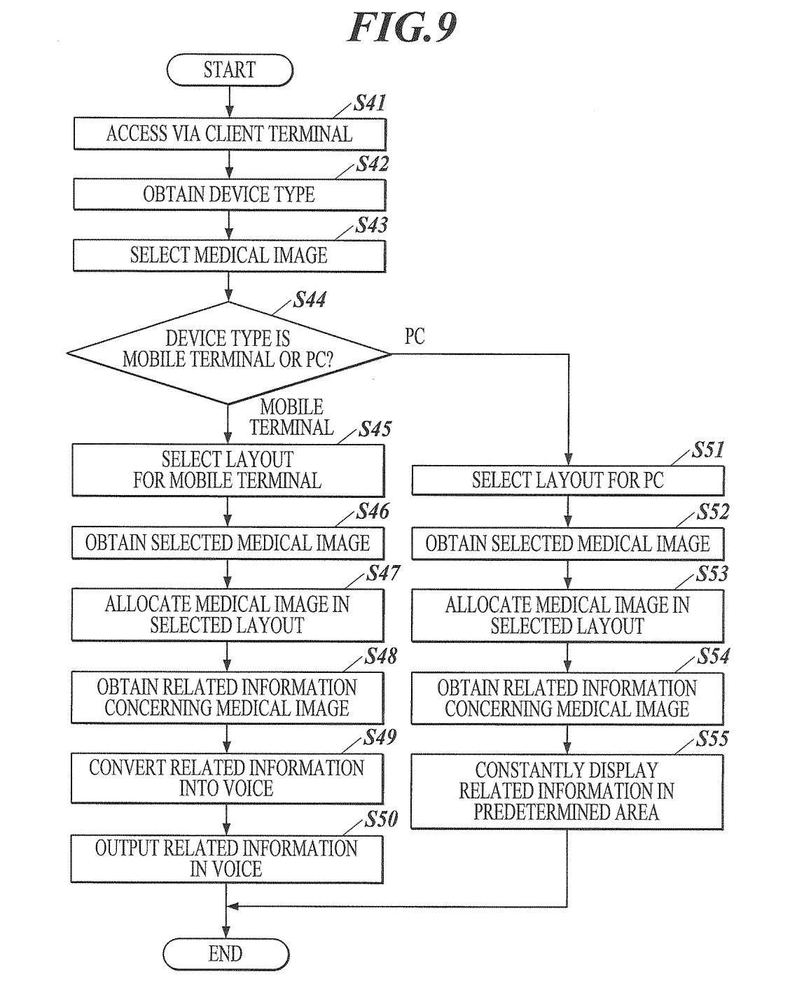

FIG. 9 is a flow chart showing the second processing of providing a medical image executed by the medical image management device 30. The processing is realized through the software processing of the CPU of the controller 31 in cooperation with the software programs stored in the ROM.

The processing at Steps S41 to S48 is identical to the processing at Steps S21 to S28 in the first processing of providing a medical image (see FIG. 5). Thus, the explanation is omitted.

At Step S47, the controller 31 provides the screen data for displaying the medical image display screen for the client terminal 50 (mobile terminal) via the communication unit 32.

In the client terminal 50 (mobile terminal), the medical image display screen is displayed on the display 53. On the medical image display screen, the medical image is shown.

After Step S48, the controller 31 generates the voice data by converting the character strings of the related information into voice (Step S49).

The controller 31 sends the generated voice data to the client terminal 50 (mobile terminal) via the communication unit 32, and causes the voice output unit 54 of the client terminal 50 to output the voice (Step S50).

In the client terminal 50 (mobile terminal), the related information is output in a voice by the voice output unit 54 on the basis of the voice data received from the medical image management device 30.

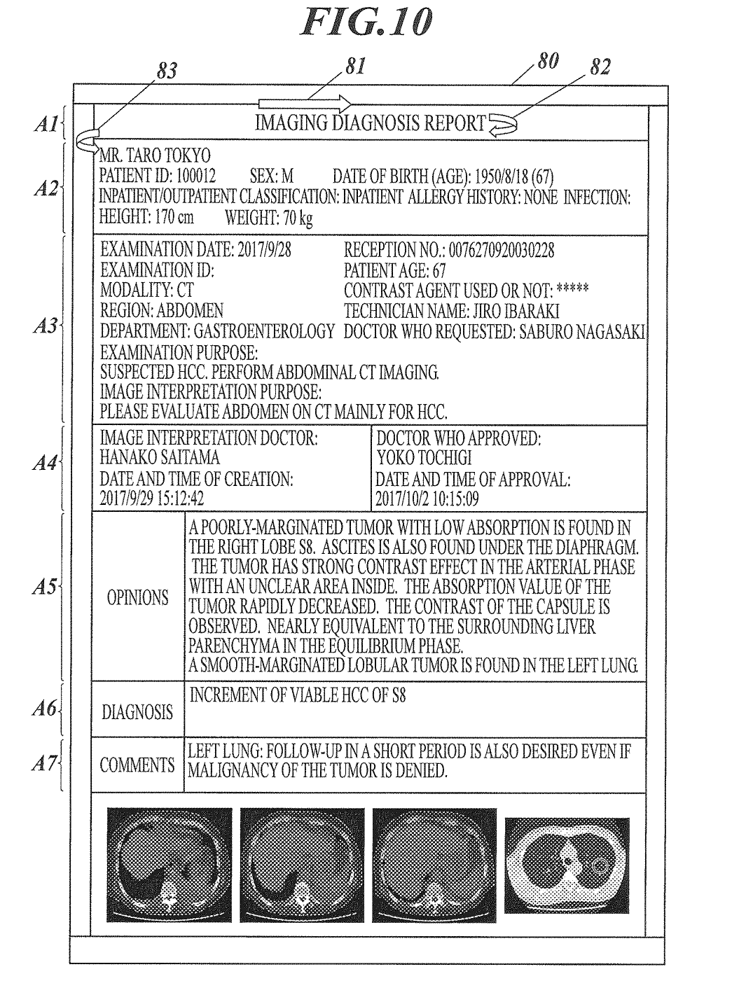

FIG. 10 is an example of the past image interpretation report (imaging diagnosis report) 80. The controller 31 generates the voice data for the information in the past image interpretation report 80, such that each word in the character string of the top row is converted into voice from left to right (along the arrow 81), and that, after reaching the last word of the row (arrow 82) and moving to the next row (arrow 83), each word in the character string of the next row is continuously converted into voice from the left edge.

The position of start and end of the voice output (text to speech) or the item of the image interpretation report to be read may be set for each facility or user, and the voice output method of the image interpretation report may be switched depending on the medical facility where the users belong or depending on the user. The areas A1 to A7 shown in FIG. 10 are successively output in a voice, but alternatively, the items may be output in a voice in a predetermined order.

At Step S44, if the device type is PC (Step S44; PC), the processing moves to Step S51.

The processing at Steps S51 to S55 is identical to the processing at Steps S30 to S34 in the first processing of providing medical images (see FIG. 5). Thus, the explanation is omitted.

After Step S50 or S55, the second processing of providing a medical image is finished.

As described, according to the second embodiment, in a case where the client terminal 50 is a mobile terminal, the transmission target part of the related information concerning the medical image is varied over time. The space for displaying the related information is not necessary. Accordingly, the medical image may be displayed in a sufficient size on the client terminal 50 while the related information concerning the medical image may be efficiently transmitted at the same time.

Specifically, in a case where the client terminal 50 is a mobile terminal, the character strings of the related information is converted into voice to generate the voice data and, on the basis of the voice data, the voice is output by the voice output unit 54 of the client terminal 50. Thus, the related information may be transmitted without the display of medical image being obstructed.

For the privacy protection of the patients, in the voice output from the client terminal 50 (mobile terminal), the information may be prevented from being leaked to the outside with a limitation being imposed. For example, the voice output of the related information may be allowed only by earphones.

The above description in the embodiments is merely an example of the medical image management device according to the present invention, and is not intended to limit the invention. The detailed configuration or operation may be suitably modified without departing from the scope of the invention.

For example, in the above embodiments, the screen layout data (layout for mobile terminal, layout for PC) may be selected depending on the device type. Alternatively, the user may select the screen layout data from multiple kinds of screen layout data prepared beforehand at the time of login from the client terminal 50.

In the examples disclosed above, a ROM is used as a computer-readable medium storing the programs for executing various kinds of processing therein, though not limited thereto. A non-volatile memory such as a flash memory or a portable recording/storage medium such as a CD-ROM may be used as an alternative computer-readable medium. Further, as a medium to provide data of the programs via communication network, a carrier wave may be used.

Although embodiments of the present invention have been described and illustrated in detail, the disclosed embodiments are made for purposes of illustration and example only and not limitation. The scope of the present invention should be interpreted by terms of the appended claims.

What is claimed is:

1. A medical image management device comprising a hardware processor that causes a mobile terminal to display a medical image thereon and transmits related information concerning the medical image via the mobile terminal at least in a part of a time period when the medical image is being displayed on the mobile terminal,
wherein the hardware processor varies a transmission target part of the related information over time.

2. The medical image management device according to claim 1, wherein the hardware processor causes a character string of the related information to be displayed with scrolling in a predetermined area on a screen for displaying the medical image in the mobile terminal.

3. The medical image management device according to claim 1, wherein the hardware processor generates voice data by converting the character string of the related information into voice and causes a voice output unit of the mobile terminal to output voice on the basis of the voice data.

4. The medical image management device according to claim 1, wherein the related information includes attached information concerning the medical image, clinical information on a patient concerning the medical image, management information on the patient concerning the medical image, an instruction of a doctor concerning the medical image, a treatment plan of the patient concerning the medical image, or an image interpretation report created on a past examination corresponding to an examination concerning the medical image.

5. A non-transitory computer-readable recording medium in which a program is stored, wherein the program causes a computer to cause a mobile terminal to display a medical image thereon, to transmit related information concerning the medical image via the mobile terminal at least in a part of a time period when the medical image is being displayed on the mobile terminal, and to vary a transmission target part of the related information over time.
